(19) United States
(12) Patent Application Publication
(10) Pub. No.: US 2022/0404333 A1
(43) Pub. Date: Dec. 22, 2022

(54) In-Line Urinalysis System and Method

(71) Applicant: C. R. Bard, Inc., Franklin Lakes, NJ (US)

(72) Inventors: Sudhakar Jagannathan, Alpharetta, GA (US); Ellena Augh, Suwanee, GA (US); Allanda Kriener, Atlanta, GA (US); Brett R. Skelton, Louisville, CO (US); Lauren Valdes, Lafayette, CO (US)

(21) Appl. No.: 17/846,860

(22) Filed: Jun. 22, 2022

Related U.S. Application Data
(60) Provisional application No. 63/213,520, filed on Jun. 22, 2021.

(51) Int. Cl. G01N 33/493; G01N 21/51; A61B 5/1459; A61F 5/44; A61M 1/00; A61M 25/00

(52) U.S. Cl. CPC G01N 33/493; G01N 21/51; A61B 5/1459; A61F 5/44; A61M 1/70; A61M 25/00

(57) ABSTRACT

Disclosed herein is a system for performing urinalysis of transurethral patients. The system includes a tubing set to receive urine from a urethral catheter. A detector assembly is operatively coupled between the tubing set and a urinalysis module coupled. The system can perform urinalysis of a urine sample disposed within the tubing set and render urinalysis information on a display of the module. Also disclosed is a method of performing urinalysis that can include operations of: (i) placing a urine sample within a cuvette of a urinalysis system, the cuvette including a lumen extending between an inlet and an outlet; (ii) projecting coherent light into the sample; (iii) collecting output light exiting the sample; (iv)extracting urinalysis data from the collected light; and (v) rendering urinalysis results on a display of the system.

### PRIORITY

This application claims the benefit of priority to U.S. Provisional Application No. 63/213,520, filed Jun. 22, 2021, which is incorporated by reference in its entirety into this application.

### BACKGROUND

Current urinalysis methods are generally labor intensive and time consuming. In typical instances, a urine sample is manually dispensed into a container. The urine sample may be manually dispensed by the patient or the sample may be manually extracted from a catheter drainage tubing set by the clinician. The sample container is typically sent to a laboratory where the urine sample is analyzed. Because of the time and effort required to obtain the sample and the time delay in getting the urinalysis results back from the laboratory, clinical decisions may be made in the absence of urinalysis results. As such, it would be advantageous for a clinician to obtain urinalysis results in a more expeditious fashion especially for transurethral patients. Accordingly, disclosed herein are systems and methods that automatically perform urinalysis for transurethral patients.

### SUMMARY OF THE INVENTION

Briefly summarized, disclosed herein is a system for performing urinalysis of transurethral patients. The system includes a tubing set configured to receive urine from a urethral catheter. A detector assembly is operatively coupled between the tubing set and a urinalysis module. The system is configured to perform urinalysis of a urine sample disposed within the tubing set and render urinalysis information on a display of the module.

In use, the tubing set is coupled with the catheter to establish urine drainage from the patient. The tubing set includes a detection chamber configured to contain the urine sample and the chamber is disposed in line with the tubing set. In some embodiments, the tubing set comprises a pair of diverter valves configured to selectively: 1) direct urine flow through the chamber when actuated to a first state, and 2) direct urine flow through a chamber-bypass tube when actuated to an alternative second state.

In some embodiments, the chamber is a tubular cuvette defining a lumen having an inlet and an outlet, and the chamber may include transparent parallel walls disposed on opposite sides of the lumen.

In some embodiments, the detector assembly includes a light source configured to project a coherent light beam through the urine sample within the chamber. The detector assembly may also include a light receiver configured to collect light exiting the urine sample and provide electrical signals to the module according to characteristics of the collected light.

The electrical signals may include scattering signals defined in accordance with dynamic light scattering of the light beam, and the scattering signals may correspond to sizes of suspended particles within the sample.

The electrical signals may include color signals defined by refracted light exiting the sample. The color signals correspond to a color composition of the urine sample, and the color composition may correspond to an osmolality of the urine sample.

The detector assembly may be selectively attachable to and detachable from the tubing set. In some embodiments, the detector assembly includes a detector housing configured to shield the light receiver from external light, and the detector housing may be selectively attachable to and detachable from the detector assembly. The detector housing may include a latch configured to secure the detector housing in a closed state.

The detector assembly may include a urine sensor configured to provide an electrical signal to the module indicating the presence and/or absence of urine within the chamber, and in some embodiments, the urine sensor includes a pressure transducer.

The system includes a console including one or more processors and a non-transitory computer-readable medium having stored thereon logic that, when executed by the one or more processors, is configured to perform operations. The operations may include commencing urinalysis upon an indication from the urine sensor of the presence of urine within the chamber and ceasing urinalysis upon an indication from the urine sensor of the absence of urine within the chamber.

The operations may further include commencing urinalysis upon an indication from the urine sensor that urine is flowing through the chamber and ceasing urinalysis upon an indication from the urine sensor that urine is not flowing through the chamber.

The operations may also include ceasing urinalysis upon completion of a defined set of urinalysis processes.

The operations may include correlating the color composition with the CIE L*a*b* color space.

The operations may include extracting a particle size distribution from the scattering signals. The operations may further include processing the particle size distribution to identify one or more of proteins, albumin, bacteria, red blood cells, white blood cells, crystals, or sediments.

In some embodiments, the operations include comparing a characteristic value of the urinalysis with an alarm limit stored in memory and as a result of the comparison, generating an alarm if the characteristic value exceeds the alarm limit.

The operations may further include comparing a characteristic value of the urinalysis with an expected value range stored in memory and as a result of the comparison, providing a notification to the user that urine collection and comprehensive testing is recommended if the measured value is outside of the expected range.

In some embodiments, the operations include adjusting an intensity of the light beam, and in some embodiments, the detector assembly is coupled with the module via a wireless connection.

In some embodiments, the module is communicatively coupled with a heart rate monitor, a blood pressure monitor, and/or a pulse oximeter, and the operations further include rendering a heart rate, a blood pressure, and/or an oxygen saturation level on the display.

Also disclosed here in is a method of performing urinalysis. The method comprises: (i) placing a urine sample within a cuvette of a urinalysis system, the cuvette having a lumen extending between an inlet and an outlet; (ii) projecting coherent light into the sample; (iii) collecting output light exiting the sample; (iv) extracting urinalysis data from the collected light; and (v) rendering urinalysis results on a display of the system.

The method may further include fluidly coupling the cuvette with a urine drainage catheter. In some embodiments, the urine sample is in motion.

Collecting output light may include collecting the output light according to dynamic light scattering techniques, and may include collecting coherent light modified by a refractive index of the urine sample.

Extracting urinalysis data may include determining a size distribution of particles within the sample and may also include determining a color composition of the sample. Rendering urinalysis results may include displaying the color composition of the sample in accordance with the CIE L*a*b* color space.

These and other features of the concepts provided herein will become more apparent to those of skill in the art in view of the accompanying drawings and following description, which describe particular embodiments of such concepts in greater detail.

### BRIEF DESCRIPTION OF DRAWINGS

A more particular description of the present disclosure will be rendered by reference to specific embodiments thereof that are illustrated in the appended drawings. It is appreciated that these drawings depict only typical embodiments of the invention and are therefore not to be considered limiting of its scope. Example embodiments of the invention will be described and explained with additional specificity and detail through the use of the accompanying drawings in which:

FIG. 1 illustrates a urinalysis system coupled with a drainage catheter, in accordance with some embodiments;

FIG. 2 is a detailed illustration of a sampling chamber of a first embodiment of the tubing set of FIG. 1, in accordance with some embodiments;

FIG. 3 is a detailed illustration of a sampling chamber of a second embodiment of the tubing set of FIG. 1, in accordance with some embodiments;

FIG. 4 is a detailed illustration of the detector assembly of the FIG. 1, in accordance with some embodiments;

FIG. 5 is a block diagram of the console of FIG. 1, in accordance with some embodiments;

FIG. 6A is a graph representing a distribution of smaller sized particles within a urine sample, in accordance with some embodiments;

FIG. 6B is a graph representing a distribution of larger sized particles with a urine sample, in accordance with some embodiments; and

FIGS. 7A and 7B are exemplary patient information display windows, in accordance with some embodiments.

### DETAILED DESCRIPTION

Before some particular embodiments are disclosed in greater detail, it should be understood that the particular embodiments disclosed herein do not limit the scope of the concepts provided herein. It should also be understood that a particular embodiment disclosed herein can have features that can be readily separated from the particular embodiment and optionally combined with or substituted for features of any of a number of other embodiments disclosed herein.

Regarding terms used herein, it should also be understood the terms are for the purpose of describing some particular embodiments, and the terms do not limit the scope of the concepts provided herein. Ordinal numbers (e.g., first, second, third, etc.) are generally used to distinguish or identify different features or steps in a group of features or steps, and do not supply a serial or numerical limitation. For example, “first,” “second,” and “third” features or steps need not necessarily appear in that order, and the particular embodiments including such features or steps need not necessarily be limited to the three features or steps. Labels such as “left,” “right,” “top,” “bottom,” “front,” “back,” “upward,” “downward,” and the like are used for convenience and are not intended to imply, for example, any particular fixed location, orientation, or direction. Instead, such labels are used to reflect, for example, relative location, orientation, or directions. Singular forms of “a,” “an,” and “the” include plural references unless the context clearly dictates otherwise. Also, the words “including,” “has,” and “having,” as used herein, including the claims, shall have the same meaning as the word “comprising.”

The terms “or” and “and/or” as used herein are to be interpreted as inclusive or meaning any one or any combination. As an example, “A, B or C” or “A, B and/or C” mean “any of the following: A; B; C; A and B; A and C; B and C; A, B and C.” An exception to this definition will occur only when a combination of elements, components, functions, steps or acts are in some way inherently mutually exclusive.

The directional terms “proximal” and “distal” are used herein to refer to opposite locations on a medical device. The proximal end of the device is defined as the end of the device closest to the end-user when the device is in use by the end-user. The distal end is the end opposite the proximal end, along the longitudinal direction of the device, or the end furthest from the end-user.

Unless defined otherwise, all technical and scientific terms used herein have the same meaning as commonly understood by those of ordinary skill in the art.

Any methods disclosed herein include one or more steps or actions for performing the described method. The method steps and/or actions may be interchanged with one another. In other words, unless a specific order of steps or actions is required for proper operation of the embodiment, the order and/or use of specific steps and/or actions may be modified. Moreover, sub-routines or only a portion of a method described herein may be a separate method within the scope of this disclosure. Stated otherwise, some methods may include only a portion of the steps described in a more detailed method.

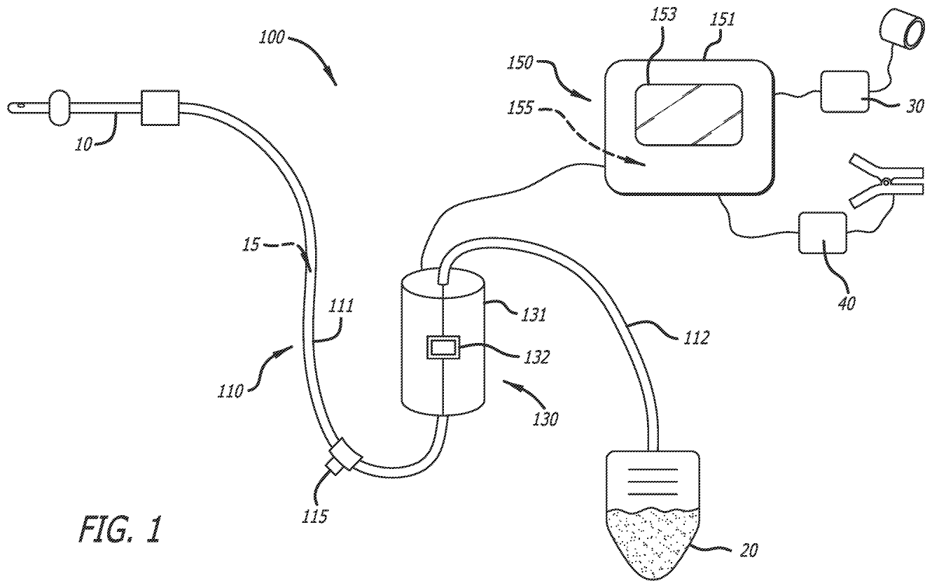

FIG. 1 illustrates a system 100 for performing urinalysis during urine drainage from a patient. The system 100 may be configured for simultaneously performing a urine drainage process and a urinalysis within a clinical setting or non-clinical setting (e.g., a home). As such, a user of the system 100 may be a trained clinician, the patient, or some other person. The system 100 generally includes a tubing set 110, a detector assembly 130, and a urinalysis module 150. The detector assembly 130 is operatively coupled with the tubing set 110 and provides electrical signals to the urinalysis module 150.

The tubing set 110 is coupled with a catheter 10 at a distal end and a collection container (bag) 20 at a proximal end. A distal tubing segment 111 extends between the catheter 10 and the detector assembly 130, and a proximal tubing segment 112 extends proximally away from the detector assembly 130 to the collection container 20. In use, urine 15 flows through the distal segment 111, through the detector assembly 130, through the proximal segment 112, and into the collection container 20. A sample port 115 provide access to the urine 15 so that a sample of the urine 15 may be extracted from the tubing set 110.

The components of the detector assembly 130 are enclosed within a housing 131 which includes a latch 132. In some embodiments, the components of the detector assembly 130 may be attached to the housing 131. The latch 132 provides for securement of the housing 131 in a closed state. The housing 131 may be selectively coupled to and decoupled from the detector assembly 130 and/or the tubing set 110. The housing 131 may be configured to shield one or more components of the detector assembly from external light. In use, the user may attach the detector assembly 130 to the tubing set 110 prior to initiating a urinalysis procedure and remove the detector assembly 130 from the tubing set 110 after the urinalysis procedure is complete.

The urinalysis module 150 includes a console 155 and may include or otherwise be coupled with a display 153. The display 153 may also include a graphical user interface (GUI). In use, the user may operate the system 100 via interaction with the GUI and view urinalysis results on the display 153.

In some embodiments, the system 100 may be coupled with blood pressure monitor 30, including a heart rate monitor, and a pulse oximeter 40 so that the patient's blood pressure, heart rate, and/or oxygen saturation information may be rendered on the display 153. In some embodiments, the blood pressure, heart rate and/or oxygen saturation data may be recorded for later correlation with urinalysis data.

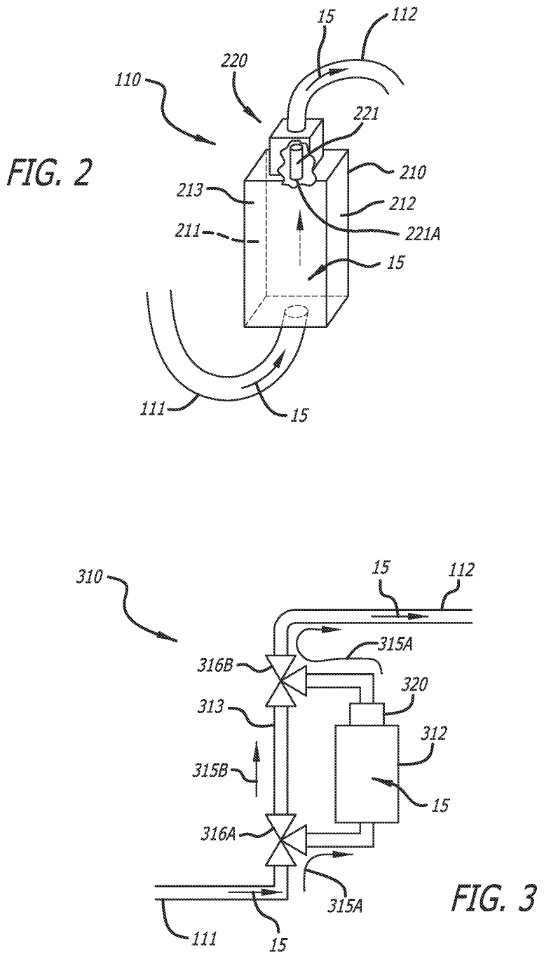

FIG. 2 is a detailed illustration of a detector portion of the tubing set 110. The tubing set 110 includes a detection chamber 212 coupled between the distal and proximal tubing segments 111, 112 so that, in use, the urine 15 flows through a chamber lumen 213. In the illustrated embodiment, the chamber 212 may generally be oriented so that the urine 15 flows vertically upward through the chamber lumen 213. In other embodiments, the urine 15 may flow horizontally or downward. The chamber 212 includes transparent side walls 211, 212 disposed opposite each other across the chamber lumen 213 to define a cuvette. In some embodiments, the side walls 211, 212 may be planar and parallel to each other. Generally speaking, the system 100 is configured to obtain a urinalysis of urine 15 disposed within the chamber 212. In some embodiments, the urinalysis may be performed while the urine 15 is flowing through the chamber 212, i.e., while the urine 15 is in motion. In other embodiments, the urinalysis may be performed while the urine 15 is at rest within the chamber 212.

A urine sensor 220 is coupled with the chamber 212. The urine sensor 220 may be configured to provide electrical signals to the module 150 pertaining to the presence and/or absence of urine 15 within the chamber 212. For example, when the chamber 212 is full of urine 15, the sensor 220 may provide a signal to the urinalysis module 150 that a sample of urine is in place and that urinalysis of the sample can be performed. Conversely, when the chamber 212 contains an insufficient volume of urine 15, the sensor 220 may provide a signal to the urinalysis module 150 that a sample of urine is not in place and that urinalysis of the sample cannot be performed.

The urine sensor 220 may include any suitable transducer and/or accommodate any suitable method for determining the presence of urine 15 within the chamber 212. In one exemplary embodiment, the urine sensor 220 may include a pressure transducer 221 configured to detect a pressure change at a working end 221A. The pressure transducer 221 is vertically oriented and positioned at a top end of the chamber 212 so that the working end 221A is disposed within the chamber 212. When a level of urine 15 is below the working end 221A (i.e., the working end 221A is not in contact with the urine), the pressure transducer 221 measures a first pressure indicating insufficient urine volume in the chamber 212. Conversely, when the level of urine 15 is above the working end 221A (i.e., the working end 221A is disposed within the urine 15) contact of the urine 15 exerts a pressure on the working end 221A defining a second measured pressure thereby indicating a sufficient volume of urine 15 within the chamber 212. As may be appreciated by one of ordinary skill, other fluid detection methods may be employed to provide the first and second signals pertaining to urine within the chamber 212 and are thus included herein.

In some embodiments, the urine sensor 220 may be configured to detect a fluid motion within the chamber 212. In such an embodiment, the urine sensor 220 may provide signals to the module 150 indicating flow or motion of urine 15 through the tubing set 110 including the chamber 212. In further embodiments, the urine sensor 220 may provide signals to the module 150 pertaining to quantified volumetric flow rate of the urine 15.

FIG. 3 is a detailed illustration of a detector portion of a second embodiment of a tubing set 310. The tubing set 310 includes a chamber 312 and a urine sensor 320 that may some respects resemble the chamber 212 and urine sensor 220 of FIG. 2. The tubing set 310 is configured to isolate a sample of urine 15 within the chamber 312 from the general flow path of the tubing set 310. The tubing set 310 includes a pair of diverter valves 316A, 316B configured to selectively direct the flow of urine 15 through the chamber 312 or through a bypass tube 313. In other words, when the diverter valves 316A, 316B are in a first actuated state, flow of urine 15 is directed through the chamber 312 as indicated by the arrows 315A. In a second alternative actuated state, the diverter valves 316A, 316B direct the flow urine 15 through the bypass tube 313 as indicated by the arrow 315B.

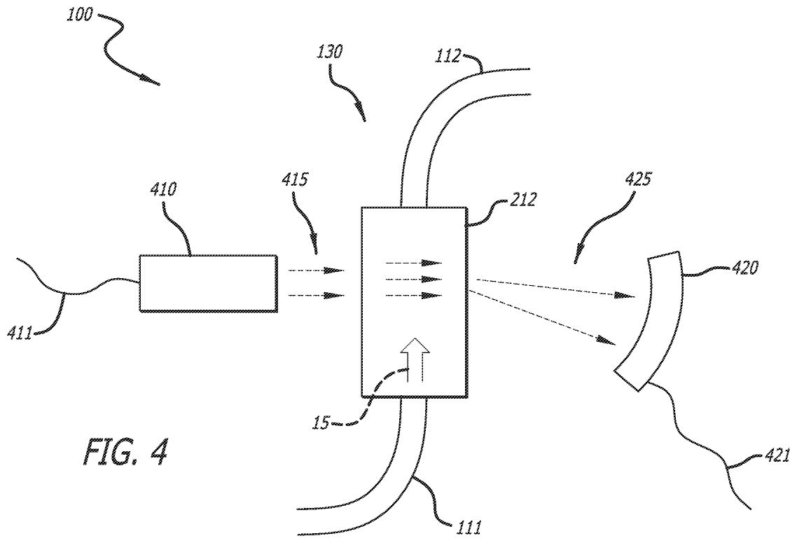

FIG. 4 illustrates the components and functionality of the detector assembly 130 of the system 100. The detector assembly 130 generally includes a light source 410 and a light receiver 420. In use, input light 415 is directed through the chamber 212 including the sample of urine 15 therein. Upon exiting the chamber 212, output light 425 is received by the light receiver 420. The light source 410 receives electrical power from the urinalysis module 150 via a wired connection 411, and the light receiver 420 delivers electrical signals to the urinalysis module 150 over a wired connection 421. In alternative embodiments, the detector assembly 130 may include a console (not shown) including power source and wireless modules so that the detector assembly 130 may be wirelessly coupled with the urinalysis module 150.

The light source 410 may comprise a laser device (e.g., a laser diode) to generate the input light 415 in the form of a coherent light beam. The light source 410 may be activated and deactivated by the urinalysis module 150. In some embodiments, the intensity and/or other parameters of the input light 415 may also be adjusted or modified by the urinalysis module 150.

The light receiver 420 may be configured to provide electrical signals in accordance with multiple modalities of the output light 425, including color detection, absorption, scattering, fluorescence, etc. The light receiver 420 includes photodetectors which may include color detectors, photomultipliers, photodiodes, phototransistors, spectrophotometers, and the like.

The light receiver 420 is configured to provide electrical signals according to dynamic light scattering (DSL) techniques. Via DSL, the electrical signals may correlate to particle concentrations and particle size distributions. The light receiver 420 is configured to provide electrical signals according to the refractive index of the urine sample. The refractive index may correlate to a color of the urine sample.

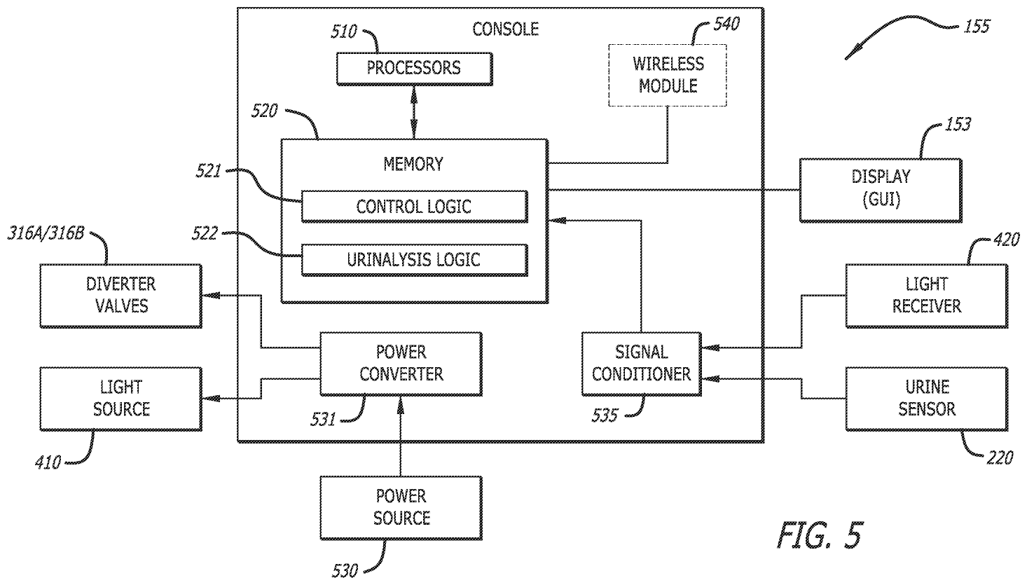

FIG. 5 is a block diagram of an exemplary console 155 included within the urinalysis module 150 of the system 100. The console 155 is powered by an external power source 530. A power converter 531 provides power at the appropriate voltages for each of the console components and the external components, including the diverter valves 316A, 316B, the urine sensor 220, the light source 410, and the light receiver 420.

The console 155 includes one or more processors 510 configured to perform operations as defined by the control logic 521 and urinalysis logic 522 stored in memory 520, the memory 520 including a non-transitory computer-readable storage medium.

A signal conditioner 535 process electrical signals from the urine sensor 220 for processing by the control logic 521. The signal conditioner 535 also processes electrical signals from the light receiver 420 for data processing by the urinalysis logic 522.

In some embodiments, the console 155 may include a wireless module 540 to facilitate wireless communication with external devices such an external display, a facility network, a personal computing device (e.g., a cell phone), the blood pressure monitor 30, and/or the pulse oximeter 40. In some embodiments, the wireless module 540 may facilitate wireless communication between the detector assembly 130 and the urinalysis module 150.

The control logic 521 is configured to perform control operations when executed by the one or more processors 510. The control operations may generally relate to operation of the system 100. In some embodiments, the control operations may include activating/deactivating the light source 410. For example, the control logic 521 may activate the light source 410 and/or the light receiver 420 upon receiving a signal from the urine sensor 220 that sufficient urine 15 is present in the chamber 212. Similarly, the control logic 521 may deactivate the light source 410 and/or the light receiver 420 upon receiving a signal from the urine sensor 220 that insufficient urine 15 is present in the chamber 212.

In a similar fashion, the control logic 521 may activate the light source 410 and/or the light receiver 420 upon receiving a signal from the urine sensor 220 that urine 15 is flowing through the chamber 212. Similarly, the control logic 521 may deactivate the light source 410 and/or the light receiver 420 upon receiving a signal from the urine sensor 220 that urine 15 is not flowing (i.e., at rest) through in the chamber 212.

In some embodiments, the control logic 521 may activate the light source 410 and/or the light receiver 420 in accordance with a predefined urinalysis schedule. In a similar fashion, the control logic 521 may deactivate the light source 410 and/or the light receiver 420 after a defined set of analysis processes are completed.

In some embodiments, the control operations may include activating/deactivating the diverter valves 316A, 316B. For example, the control logic 521 may activate the 316A, 316B to cause urine 15 to flow into the chamber 312. The control logic 521 may then deactivate the valves 316A, 316B to trap a sample of urine 15 within the chamber 312. After completion of defined urinalysis processes, the control logic 521 may activate the 316A, 316B to release the sample of urine 15 from the chamber 312 and allow a subsequent sample of urine 15 to enter the chamber 312.

The urinalysis logic (herein after logic) 522 is configured to perform analysis operations when executed by the one or more processors 510. More specifically, the logic may process conditioned signal data from the detector assembly 130. The analysis operations may generally facilitate clinical assessment of a patient's health status based upon detected conditions of the patient's urine, such as kidney and liver health, for example.

The analysis operations may include receiving scattering data pertaining to the distribution and sizes of the particles detected within the urine 15. The operations may include the DSL processing of the scattering data to determine sizes of particles.

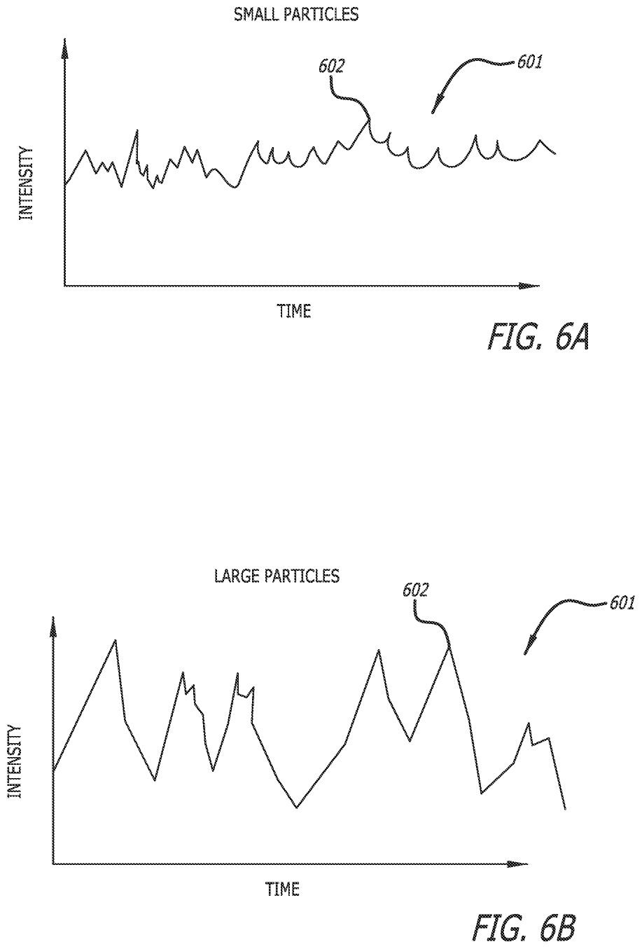

FIGS. 6A and 6B illustrate exemplary particle size graphs showing results of particle size detection within a flowing urine sample through the chamber 212 (FIG. 2) as may be rendered on the display 153 (FIG. 1). The graphs indicate an intensity along the y-axis and time along the x-axis. The intensity may provide a visual indication of a size of the particles. For example, each peak 602 along the trend line 601 may represent a detected particle, and the intensity (i.e., height) of the peak 602 may represent the size of the respective particle. Similarly, the number of peaks 602 along the trend line 601 may represent a concentration or distribution of the particles within the flowing sample. FIG. 6A shows an exemplary trend line as may be defined from a flowing urine sample containing a relatively high concentration of particles having smaller sizes. Conversely, FIG. 6B shows an exemplary trend line as may be defined from a flowing urine sample containing a lower concentration of particles having larger sizes.

The operations may further include deriving types of particles from the scattering data, such as proteins (albumin), epithelial cells, red blood cells, white blood cells, crystals, and casts, for example. As the size of the exemplary particles may be related to the type of particle, the operations of the logic 522 may determine the types of various detected particles within the urine 15 based on defined a size range. In some embodiments, the operations may include determining a concentration of one or more types of particles. By way of example, the logic 522 may determine a concentration red blood cells within the urine 15.

The analysis operations may also include comparing a determined concentration of particles within a defined size range, including high and/or low limits. For example, the memory 520 may include (i.e., stored thereon) an expected range of albumin within the urine of a typical healthy patient. The logic 522 may then compare a determined concentration of albumin within the urine 15 with the expected healthy range of albumin. As a result of the comparison, the logic 522 may provide a notification to the user if the determined concentration is outside the expected range. Such a notification may include visual information or indicia rendered on the display (e.g., a recommended course of action) and/or an audio alarm.

The analysis operations may include determining a color composition of the urine 15 from color composition data received from the detector assembly 130. The logic 522 may process the color composition data for correlation with a subjective color scale. In some embodiments, the color composition data may be processed to correlate with a tristimulus colorimetry developed by the International Commission on Illumination generally referred to as the “CIE L*a*b*” color space, which is illustrated and discussed in “The Effect of Hydration on Urine Color Objectively in CIE L*a*b* Color Space,” Frontiers in Nutrition, October 2020.” The CIE L*a*b* space is three-dimensional, and covers the entire range of human color perception. It is based on the opponent color model of human vision, where red/green forms an opponent pair, and blue/yellow forms an opponent pair. The lightness value (L*) defines black at zero and white at 100. The “a” axis is relative to the green-red opponent colors, with negative values toward green and positive values toward red. The “b” axis represents the blue-yellow opponents, with negative numbers toward blue and positive toward yellow.

Studies of shown a correlation between urine color and osmolality. An osmolality increase in urine has been correlated with an increase in “b” value (i.e., a more yellow color). An osmolality increase also correlates with a darker urine (a decrease in “L*” values). It has been shown, osmolality can be correlated with a change in color along the green-red axis. Slight increases in osmolality have been shown to correlate with a decrease in “a” values indicating a green hue, while further increased osmolality correlates with increased “a” values.

As such, the logic 522 may include correlation algorithms relating to the CIE L*a*b*” color space. The logic 522 may then apply the algorithms to color composition data received from the detector assembly to determine an osmolality of the urine sample.

In some embodiments, the analysis operations may include receiving urine flow rate data from the urine sensor 220. The logic 522 may integrate the flow rate data over time during the drainage process to track a volume of urine 15 within the collection container 20.

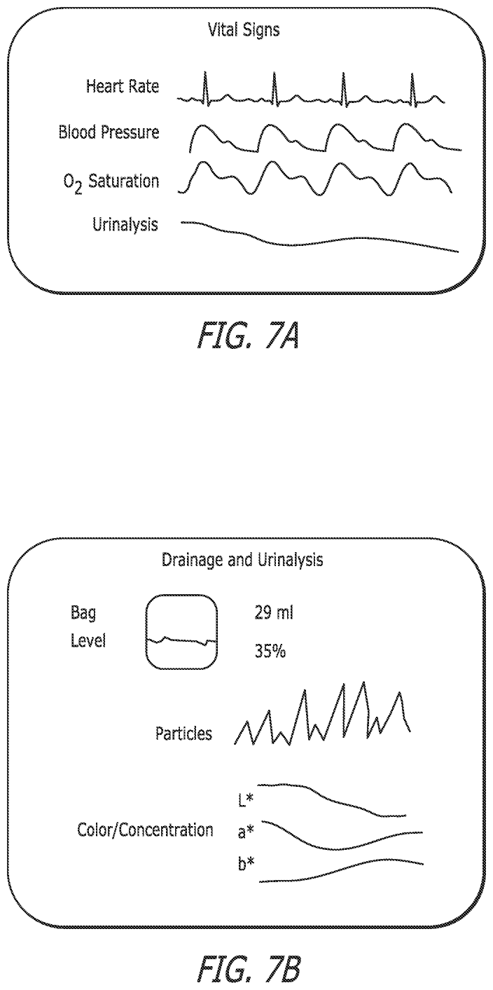

The analysis operations include rendering urinalysis and other information on the display 153. FIGS. 7A and 7B, illustrate exemplary windows that may be rendered on the display 153. The window FIG. 7A shows an exemplary set of vital signs for which observation may be desirable including heart rate, blood pressure, oxygen (O₂) saturation, and urinalysis. The vital signs may be rendered in a real-time auto-scrolling fashion. In other words, the vital signs may be displayed as they occur. In some embodiments, the user may be able to manually scroll through recorded historical vital sign data to observe the vital signs during an earlier period of the process. In some embodiments all or a subset of the urinalysis data may also be displayed on the vital signs window. The urinalysis data may include particle data such as the graphs of FIGS. 6A, 6B or color composition data correlated to the CIE L*a*b* color space.

The window of FIG. 7B shows other exemplary information associated with drainage process generally and/or the urinalysis for which observation may be desirable. For example, general drainage information may include an accumulated drainage volume and a percentage of the bag capacity occupied by the accumulated drainage volume. The urinalysis data may include the particle data such as the graphs of FIGS. 6A, 6B and/or the color composition data correlated to the CIE L*a*b* color space.

Embodiments of the invention may be embodied in other specific forms without departing from the spirit of the present disclosure. The described embodiments are to be considered in all respects only as illustrative, not restrictive. The scope of the embodiments is, therefore, indicated by the appended claims rather than by the foregoing description. All changes that come within the meaning and range of equivalency of the claims are to be embraced within their scope.

1. A system for performing urinalysis of a transurethral patient, comprising:
a tubing set configured to receive urine from a urine drainage catheter;
a detector assembly operatively coupled with the tubing set; and
a urinalysis module coupled with the detector assembly, wherein the system is configured to perform urinalysis of a urine sample disposed within the tubing set.

2. The system according to claim 1, wherein the module is configured to render urinalysis information on a display.

3. The system according to claim 1, wherein, in use, the tubing set is coupled with a catheter inserted into the patient.

4. The system according to claim 1, wherein the tubing set comprises a chamber configured to contain the urine sample.

5. The system according to claim 1, wherein the chamber is disposed in line with the tubing set.

6. The system according to claim 1, wherein the tubing set comprises a pair of diverter valves configured to selectively direct urine flow through:
the chamber when actuated to a first state, and
a chamber-bypass tube when actuated to an alternative second state.

7. The system according to claim 1, wherein chamber is a tubular cuvette defining a lumen having an inlet and an outlet.

8. The system according to claim 1, wherein the chamber comprises transparent parallel walls disposed on opposite sides of the lumen.

9. The system according to claim 1, wherein the detector assembly comprises:
a light source configured to project a coherent light beam through the urine sample within the chamber, and
a light receiver configured to:
collect light exiting the urine sample, and
provide electrical signals to the module in accordance with characteristics of the collected light.

10. The system according to claim 9, wherein the electrical signals include scattering signals defined in accordance with dynamic light scattering of the light beam.

11. The system according to claim 10, wherein the scattering signals correspond to sizes of suspended particles within the sample.

12. The system according to claim 9, wherein the electrical signals include color signals defined by refracted light exiting the sample.

13. The system according to claim 12, wherein the color signals correspond to a color composition of the urine sample.

14. The system according to claim 13, wherein the color composition corresponds to an osmolality of the sample.

15. The system according to claim 1, wherein the detector assembly is selectively attachable to and detachable from the tubing set.

16. The system according to claim 1, wherein the detector assembly comprises a detector housing configured to shield the light receiver from external light.

17. The system according to claim 16, wherein the detector housing is selectively attachable to and detachable from the detector assembly.

18. The system according to claim 16, wherein the detector housing comprises a latch configured to secure the detector housing in a closed state.

19. The system according to claim 1, wherein the detector assembly comprises a urine sensor configured to provide an electrical signal to the module indicating the presence and/or absence of urine within the chamber.

20. The system according to claim 19, wherein the urine sensor comprises a pressure transducer.

21. The system according to claim 1, wherein the module comprises a console including one or more processors and a non-transitory computer-readable medium having stored thereon logic that, when executed by the one or more processors, is configured to perform operations corresponding to performance of the urinalysis of the urine sample disposed within the tubing set.

22. The system according to claim 21, wherein the operations comprise:
commencing the urinalysis upon an indication from the urine sensor of the presence of the urine within the chamber; and
ceasing the urinalysis upon an indication from the urine sensor of the absence of the urine within the chamber.

23. The system according to claim 21, wherein the operations further comprise:
commencing the urinalysis upon an indication from the urine sensor that the urine is flowing through the chamber; and
ceasing the urinalysis upon an indication from the urine sensor that the urine is not flowing through the chamber.

24. The system according to claim 21, wherein the operations further comprise ceasing the urinalysis upon completion of a defined set of urinalysis processes.

25. The system according to claim 21, wherein the operations further comprise correlating the color composition with the CIE L*a*b* color space.

26. The system according to claim 21, wherein the operations further comprise extracting a particle size distribution from the scattering signals.

27. The system according to claim 26, wherein the operations further comprise processing the particle size distribution to identify one or more of proteins, albumin, bacteria, red blood cells, white blood cells, crystals, casts, or sediments.

28. The system according to claim 21, wherein the operations further comprise:
comparing a characteristic value of the urinalysis with an alarm limit stored in memory; and
as a result of the comparison, generating an alarm if the characteristic value exceeds the alarm limit.

29. The system according to claim 21, wherein the operations further comprise:
comparing a measured value of a urine characteristic with an expected value range stored in memory, and
as a result of the comparison, providing notification to the user that urine collection and comprehensive testing is recommended if the measured value is outside of the expected range.

30. The system according to claim 21, wherein the operations further comprise adjusting an intensity of the light beam.

31. The system according to claim 1, wherein the detector assembly is coupled with the module via a wireless connection.

32. The system according to claim 1, wherein:
the module is communicatively coupled with a heart rate monitor, a blood pressure monitor, and/or a pulse oximeter, and
the operations further comprise rendering a heart rate, blood pressure, and/or an oxygen saturation level on the display.

33-40. (canceled)
